(19) United States
(12) Patent Application Publication
(10) Pub. No.: US 2006/0190066 A1
(43) Pub. Date: Aug. 24, 2006

(54) System and method for bringing hypothermia rapidly onboard

(76) Inventor: William J. Worthen, Coto de Caza, CA (US)

Correspondence Address: ROGITZ & ASSOCIATES, 750 B STREET, SUITE 3120, SAN DIEGO, CA, 92101, US

(21) Appl. No.: 11/064,187

(22) Filed: Feb. 23, 2005

(51) Int. Cl. A61F 7/00; A61F 7/12

(52) U.S. Cl. 607/105; 607/113

(57) ABSTRACT

An intravenous heat exchange catheter and/or an external cooling pad can be used to maintain hypothermia in, e.g., a cardiac arrest patient, but to accelerate the cooling process the patient first can be infused with cold saline before the opportunity arises to connect the catheter or pad to the patient.

### I. FIELD OF THE INVENTION

The present invention relates generally to therapeutic hypothermia.

### II. BACKGROUND OF THE INVENTION

Intravascular catheters have been introduced for controlling patient temperature. Typically, a coolant such as saline is circulated through an intravascular heat exchange catheter, which is positioned in the patient's bloodstream, to cool or heat the blood as appropriate for the patient's condition. The coolant is warmed or cooled by a computer-controlled heat exchanger that is external to the patient and that is in fluid communication with the catheter.

For example, intravascular heat exchange catheters can be used to combat potentially harmful fever in patients suffering from neurological and cardiac conditions such as stroke, subarachnoid hemorrhage, intracerebral hemorrhage, cardiac arrest, and acute myocardial infarction, or to induce therapeutic hypothermia in such patients. Further, such catheters can be used to rewarm patients after, e.g., cardiac surgery or for other reasons. Intravascular catheters afford advantages over external methods of cooling and warming, including more precise temperature control and more convenience on the part of medical personnel.

The following U.S. patents, all of which are incorporated herein by reference, disclose various intravascular catheters/systems/methods: U.S. Pat. Nos. 6,419,643, 6,416,533, 6,409,747, 6,405,080, 6,393,320, 6,368,304, 6,338,727, 6,299,599, 6,290,717, 6,287,326, 6,165,207, 6,149,670, 6,146,411, 6,126,684, 6,306,161, 6,264,679, 6,231,594, 6,149,676, 6,149,673, 6,110,168, 5,989,238, 5,879,329, 5,837,003, 6,383,210, 6,379,378, 6,364,899, 6,325,818, 6,312,452, 6,261,312, 6,254,626, 6,251,130, 6,251,129, 6,245,095, 6,238,428, 6,235,048, 6,231,595, 6,224,624, 6,149,677, 6,096,068, 6,042,559.

Surface cooling may be less optimally used. For example, externally applied cooling pads are disclosed in U.S. Pat. Nos. 6,827,728, 6,818,012, 6,802,855, 6,799,063, 6,764,391, 6,692,518, 6,669,715, 6,660,027, 6,648,905, 6,645,232, 6,620,187, 6,461,379, 6,375,674, 6,197,045, and 6,188,930 (collectively, “the external pad patents”), all of which are incorporated herein by reference.

Regardless of the modality of cooling, it is believed that the sooner a patient is cooled after ischemic insult, the better the therapy. The present invention recognizes that many patients will have their first encounter with health care personnel in ambulances, prior to being afforded the opportunity for critical care such as controlled maintenance of hypothermia. Thus, it would be advantageous, as understood herein, to provide a means to bring cooling on board to patients as soon as possible.

### SUMMARY OF THE INVENTION

A system for controlling patient temperature includes a closed loop heat exchange catheter configured for placement in the circulatory system of a patient to exchange heat with the blood of the patient. The system also includes a source of cold fluid, with the cold fluid being colder than normal body temperature and infusable from the source into the patient without using the catheter.

The catheter may be configured for percutaneous advancement into the central venous system of the patient. The catheter can carry coolant that is not infused into the bloodstream of the patient.

In another aspect, a method for treating a patient using hypothermia includes injecting cold saline into the venous system of the patient while the patient is located in an ambulance or in an emergency room of a hospital. Then subsequently hypothermia is maintained in the patient using an external heat exchange pad or an intravascular heat exchange catheter while the patient is in an operating room of a hospital or an intensive care unit of a hospital.

In yet another aspect, a method for treating a patient includes infusing into the patient's venous system a cold fluid having a temperature lower than a temperature of the patient to cause the fluid to mix with the blood of the patient and thereby to cool the patient. The method also includes engaging a cooling apparatus with the patient to maintain a desired hypothermic condition in the patient.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a schematic diagram showing two modalities of controlled hypothermia maintenance in a patient, along with an apparatus for quickly reducing patient temperature; and

FIG. 2 is a flow chart of the present logic.

### DETAILED DESCRIPTION OF THE PREFERRED EMBODIMENT

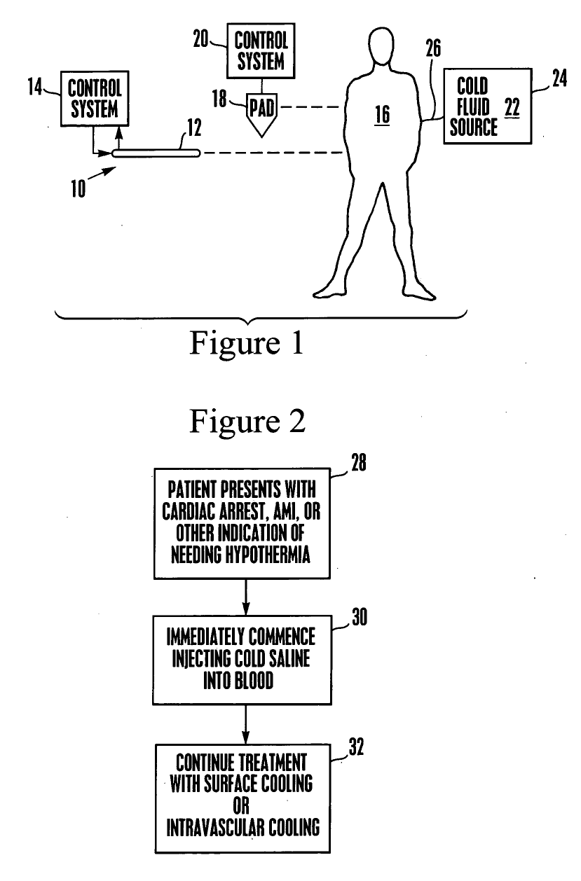

Referring initially to FIG. 1, a system is shown, generally designated 10, that includes a heat exchange catheter 12 that is in fluid communication with a catheter temperature control system 14.

In accordance with present principles, the system 10 can be used to induce therapeutic hypothermia in a patient 16 using a catheter in which coolant circulates in a closed loop, such that no coolant enters the body. While certain preferred catheters are disclosed below, it is to be understood that other catheters can be used in accordance with present principles, including, without limitation, any of the catheters disclosed in the following U.S. patents, all incorporated herein by reference: U.S. Pat. Nos. 5,486,208, 5,837,003, 6,110,168, 6,149,673, 6,149,676, 6,231,594, 6,264,679, 6,306,161, 6,235,048, 6,238,428, 6,245,095, 6,251,129, 6,251,130, 6,254,626, 6,261,312, 6,312,452, 6,325,818, 6,409,747, 6,368,304, 6,338,727, 6,299,599, 6,287,326, 6,126,684. The catheter 12 may be placed in the venous system, e.g., in the superior or inferior vena cava.

Instead of or in addition to the catheter 12, the system 10 may include one or more pads 18 that are positioned against the external skin of the patient 16 (only one pad 18 shown for clarity). The pad 18 may be, without limitation, any one of the pads disclosed in the external pad patents. The temperature of the pad 18 can be controlled by a pad controller 20 in accordance with principles set forth in the external pad patents to exchange heat with the patient 16, including to induce therapeutic mild or moderate hypothermia in the patient in response to the patient presenting with, e.g., cardiac arrest, myocardial infarction, stroke, high intracranial pressure, traumatic brain injury, or other malady the effects of which can be ameliorated by hypothermia.

To cool the patient while awaiting engagement of the catheter 12 and/or pad 18 with the patient, cold fluid 22 in a cold fluid source 24 may be injected into the patient and in particular into the patient's venous system through a pathway 26. Without limitation, the pathway 26 may an IV line, the source 24 may be an IV bag, and the fluid 22 may be chilled saline, e.g., saline at the freezing point or slightly warmer. Or, the source may be a syringe, and the saline can be injected directly into the bloodstream of the patient.

Now referring to FIG. 2, at block 28 the patient presents with symptoms indicating that the application of hypothermia is appropriate. For instance, the patient may have cardiac arrest, and may be resuscitated. Or, the patient may present with myocardial infarction or stroke or other malady.

At block 30, cold saline 22 is immediately (in the case of cardiac arrest patients, immediately after resuscitation) injected into the patient's bloodstream, preferably at a venous site, using the source 24. This can occur in, e.g., an ambulance on the way to the hospital for further temperature management, and/or in the hospital emergency room. Hypothermia treatment including the establishment and maintenance of mild or moderate hypothermia subsequently is effected at block 32 using the catheter 12 and/or pad 18, typically in the operating room or intensive care unit of a hospital, although in some hospitals the step at block 32 may begin while the patient is still in the emergency room or even while the patient is still in the ambulance.

While the particular SYSTEM AND METHOD FOR BRINGING HYPOTHERMIA RAPIDLY ONBOARD as herein shown and described in detail is fully capable of attaining the above-described objects of the invention, it is to be understood that it is the presently preferred embodiment of the present invention and is thus representative of the subject matter which is broadly contemplated by the present invention, that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more”. All structural and functional equivalents to the elements of the above-described preferred embodiment that are known or later come to be known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. §112, sixth paragraph, unless the element is expressly recited using the phrase “means for” or, in the case of a method claim, the element is recited as a “step” instead of an “act”.

I claim:

1-3. (canceled)

4. A method for treating a patient using hypothermia, comprising:
injecting cold saline into the venous system of the patient while the patient is located in at least one of: an ambulance, or an emergency room of a hospital; then subsequently
maintaining hypothermia in the patient using at least one of: an external heat exchange pad, or an intravascular heat exchange catheter, while the patient is in at least one of: an operating room of a hospital, or an intensive care unit of a hospital.

5. The method of claim 4, comprising maintaining hypothermia in the patient using an external heat exchange pad.

6. The method of claim 4, comprising maintaining hypothermia in the patient using an intravascular heat exchange catheter.

7. The method of claim 4, wherein the patient has suffered a cardiac arrest, and the method includes resuscitating the patient prior to the injecting act.

8. The method of claim 4, wherein the patient has suffered a myocardial infarction.

9. The method of claim 4, wherein the injecting act is undertaken using an IV bag connected to the venous system of the patient.

10. A method for treating a patient, comprising:
infusing into the patient's venous system a cold fluid having a temperature lower than a temperature of the patient to cause the fluid to mix with the blood of the patient and thereby cool the patient; and
engaging a cooling apparatus with the patient to maintain a desired hypothermic condition in the patient.

11. The method of claim 10, wherein the cooling apparatus is an intravascular heat exchange catheter.

12. The method of claim 10, wherein the cooling apparatus is an externally applied heat exchange pad on the skin of the patient.
